(19) United States
(12) Patent Application Publication
(10) Pub. No.: US 2022/0336083 A1
(43) Pub. Date: Oct. 20, 2022

(54) METHOD AND SYSTEM FOR DETERMINING USER WELLBEING

(71) Applicant: MINERVA GOODS LTD, TEL AVIV (IL)

(72) Inventors: DOVEV GOLDSTEIN, TEL AVIV (IL); RACHEL YARCONY GOLDSTEIN, TEL AVIV (IL)

(21) Appl. No.: 17/827,819

(22) Filed: May 30, 2022

Related U.S. Application Data
(63) Continuation-in-part of application No. PCT/IL2020/051337, filed on Dec. 28, 2020.

(30) Foreign Application Priority Data
Dec. 30, 2019 (IL) 271771

(51) Int. Cl. G16H 20/70; G16H 10/20; G16H 10/60

(52) U.S. Cl. CPC G16H 20/70; G16H 10/20; G16H 10/60

(57) ABSTRACT

A method for determining a user wellbeing profile, the method including using a processor to detect a change in physiological data of the user, based on input from a sensor unit and selecting, in accordance with the detected change in physiological data, a specific question relating to psychological data of the user, where user's wellbeing profile can be calculated based on a response from the user to the specific question which was selected in accordance with a specific change in physiological data.

### FIELD

The present invention is in the field of determining user wellbeing and personalized nutrition selection, specifically relating to selection of stress relieving nutritional additives.

### BACKGROUND

Stress, as well as physiological and psychological tension, affect various aspects of people's life. There are various techniques for relieving stress including lifestyle management, nutrition, medication, and others.

### SUMMARY

Personalization of nutrition additives enables lifestyle and stress management to be tailored and directed to different people's specific needs. The present invention provides personalization based on at least one of psychological and physiological parameters of a user and provides recommendations of selected nutrition additive items directed to relieving specific lifestyle or stress aspects. Also, in some embodiments, the present invention provides personalized recommendation on nutrition additive items based on physiological data and stress/anxiety psychological analysis and correlations between them. The invention may also enable monitoring the psychological and/or physiological parameters, and in some embodiments provide indications of consumption of the recommended nutrition additive items. This allows fine tuning and monitoring the effects of the selected nutrition additive items on each specific user for improving selection and personalization of the recommended nutrition additive items.

Generally, the present invention may be implemented by one or more computer systems, operated locally or by network communication with a remote processing utility (e.g., at a server location). The one or more computer systems are generally in communication with one or more user (the user) via a user interface (UI), and with a corresponding provisional center (typically via a communication network). The invention operates to obtain, e.g., directly from the user and/or using one or more third party databases or sensors, user personal data indicative of one or more parameters associated (separately or combined in groups of parameters) with wellbeing of the user. Based on the user personal data, the present invention operates to determine a user anxiety or wellbeing profile in accordance with a selected number of wellbeing parameters, and to provide recommendation on selected collection of nutrition additive items (edibles) having active ingredients, selected to address the user's wellbeing profile and in accordance thereto. In this connection the term wellbeing profile as used herein refers mainly to a profile of the user relating to stress parameters, however as there are various types of stress and various parameters affecting stress, it should be understood that a wellbeing profile may include various parameters associated with physiological and psychological aspects, and a combination of such aspects affecting the user.

Generally, the present invention utilizes providing user personal data comprising at least one of user psychological data and user physiological data. The psychological data may generally include user subjective data, e.g., provided by the user in response to a selected set of questions relating to wellbeing and/or stress profile of the user. Different researches, either in the field of pain management or in the field of stress management, have shown that user assessment of personal wellbeing may provide increased accuracy as compared to other tests developed by professionals. To this end the present invention may provide a user with a questionnaire formed by a collection of one or more questions associated with different stress parameters, e.g., questions on user activity, habits, feeling, issues that may disturb the user, etc.

The use of physiological and/or physical data can provide additional information relating to user wellbeing and may enable monitoring of variations at an increased resolution. To this end, the present invention may utilize one or more sensors, e.g., sensors connected to an electronic device (e.g., smartphone, activity sensor/watch or smartwatch) operating and/or providing a user interface dedicated to the present technique, or third-party sensors used by the user for monitoring personal activity. Additionally or alternatively, the present invention may obtain data from one or more database (e.g., third-party database) storing user activity data, such as sports application, meditation application, etc.

Generally, the embodiments of the invention operate to process the personal data and determine a wellbeing profile of the user. The wellbeing profile may be formed by determining a score of one or more selected wellbeing parameters. For example, a technique in one embodiment of the invention may determine a user score associated with diurnal wellbeing related parameters and nocturnal wellbeing related parameters. In some embodiments, the diurnal parameters may include comfort (contentedness), focus, and energy. In some embodiments, the nocturnal wellbeing parameters may include calmness, relaxation, and sleep quality.

Generally, stress evaluation is based on two data categories, physiological and psychological stress related parameters. Both may be used together to determine a complete wellbeing profile of the user. In this connection, while collection of physiological data may provide relatively immediate information, the physiological parameters themselves may be interpreted differently when considered in combination with psychological data of the user. To this end embodiments of the invention may preferably combine collection of physiological parameters as well as collection of psychological data of the user to accurately construct a stress related wellbeing profile. The above-described diurnal and nocturnal wellbeing parameters are associated with stress types based on Diagnostic & Statistical Manual of Mental Disorders (DSM5) relating to diagnosis of stress or anxiety disorders based on anxiety types including Restlessness (keyed up), Being easily fatigued, Difficulty concentrating, Irritability, Muscle tension and Sleep disturbance.

Accordingly, embodiments of the invention may utilize processing of the user data, including physiological and psychological data, for determining an anxiety score of the user. The anxiety score may be determined by correlating stress/anxiety levels determined based on physiological data with anxiety level and type/area determined from the psychological data. This correlation enables embodiments of the invention to characterize a user anxiety profile, and determine recommended anxiety relieving nutrition additive items (e.g., based on active ingredients therein) for the user.

To improve the user wellbeing, techniques of the invention may be used in combination with selected nutrition additive items (edibles). The nutrition additive items are prepared from a collection of nutrition items and comprise a selected profile of active ingredients for improving user wellbeing in selected parameters. To this end the nutrition additive items may be prepared using a selected composition and dosage of Terpenes (or terpenoids) and/or functional herbs. For example, the nutrition additive items may include terpenes or terpenoids selected from: a-Pinene, b-Pinene, b-Caryophyllene, Terpinolene, a-Humulene, a-Terpineol, 3A-Carene, Limonene, Linalool, a-Bisabolol, and trans-Ocimene. Additional terpenes that can be used include fenugreek (Trigonella foenum-graecum) or fenugreek extract (e.g., fenulife), and Phosphatidylserine (e.g., sharp ps). Each type of nutrition additive item may include a selected profile of ingredients (e.g., terpenes) in accordance with the effect desired to be associated with each specific type of nutrition additive item.

Additional active ingredients may comprise selected dosages of at least one of Cannabidiol (CBD) and Tetrahydrocannabinol (THC). In some embodiments, the active ingredients may comprise one or more active ingredients selected from: green tea, lemon balm, valerian, hops, guarana, chamomile, ginger, passion flower, ginseng, sage, rosemary, artichoke, oats or green oats such as Avena sativa (e.g., neuravena).

The nutrition additive items may include a number of items, having different profiles of active ingredients directed at improving selected aspects of a user wellbeing profile. For example, certain nutrition additive items may be directed at improving sleep quality, others are directed at improving relaxation or calmness, and certain additional items may be directed at improving focus, comfort, or energy of the user. Generally, embodiments of the invention determine recommendations of a selection of nutrition additive items in accordance with a wellbeing score of the user, to provide personalized wellbeing enhancement to the user. For example, in some embodiments, the user receives a recommendation on a collection of nutrition additive items to be consumed in the duration of a month (e.g., one or two items per day consumed morning and/or evening). At the beginning of each month, embodiments of the invention may update a user wellbeing profile and may generate data on a recommended selection of nutrition additive items for the coming month in accordance with wellbeing scores of the user. For example, the user wellbeing score of the different parameters may be normalized to integer numbers given a total number of, e.g., thirty nutrition additive items, and the selection of nutrition additive items is recommended based on the normalized score.

Generally, the present technique may operate periodically for determining a user wellbeing profile, at a selected time scale, e.g., each week, two weeks, months etc. Within the selected time scale, and upon determining the user wellbeing profile, a corresponding set of nutrition additive items is selected, planned to be consumed within the coming period (e.g., the next week, two weeks, month, etc.). The technique may request user preferences, such as flavor, type of nutrition additive items, medical requirements, allergies, and approval for the order, and operate for generating a provision request. The provision request is transmitted to one or more provision centers, requesting delivery of the selected set of nutrition additive items to the specific user. Such a request may generally include user details, address, and set of nutrition additive items to be delivered. The provision center is in charge of providing the requested nutrition additive items to the user using one or more selected delivery methods.

Periodic update of the user wellbeing profile, using updated physiological and/or psychological data, or utilizing additional data, not relied on in the initial stage of determining the user wellbeing profile, enables determining efficacy of the selected nutrition additive items and updating the selection of nutrition additive items to address current issues of user wellbeing. For improved monitoring of efficacy of the nutrition additive items, embodiments of the invention may further request user indication on consumption of the different nutrition additive items. Such indication may generally comprise data on type/identity of the nutrition additive item being consumed and time of consumption. This indication of consumption may be used for further processing of user physiological and psychological data collected after the consumption indication, for estimating the effect of the nutrition additive items on the user wellbeing. As some individuals may react differently to selected active ingredients, the indication of consumption may be used for processing collected personal data and determining personal effects of the nutrition additive item on the user. For example, if a user receives a recommendation on sleep quality related nutrition additive items, but the data collected after consumption of the specific items shows deteriorated sleep quality, a different collection of nutrition additive items may be preferred for this specific user.

Thus, according to a broad aspect, the present invention provides a method (e.g., computer implemented method) comprising: providing user data comprising at least one of user psychological data and user physiological data; using one or more processors for processing said user data and determining a current user anxiety/wellbeing profile comprising user score in one or more selected user anxiety/wellbeing parameters; determining recommendation on a first collection of personalized nutrition additive items (edibles) in accordance with said user anxiety/wellbeing profile; and generating provision request indicating said first collection of personalized nutrition additive items to be provided to the user and transmitting said provision request via a communication network to designated supply management utility. According to some embodiments, providing user data may comprise operating a user interface unit and generating a request for the user to provide user psychological data indicative of at least one of user behavior and personal preferences.

According to some embodiments, the request may comprise providing the user with a selected series of questions associated with at least one of user behavior, personal preferences, and feelings.

According to some embodiments, providing user data may comprise providing user physiological data indicative of at least one of user biomedical parameters and user activity. Providing user data may also comprise, operating one or more sensor units for monitoring said user physiological data. Additionally or alternatively, providing user data may comprise obtaining data indicative of user physiological data obtained from one or more activity related programs. Further, providing user data may comprise obtaining video data including a user's face and processing said video data in accordance with one or more user behavior parameters selected from at least one of eye movement and facial expressions.

According to some embodiments, providing user data may also comprise obtaining audio data including a user's voice, and processing said audio data in accordance with one or more parameters comprising voice pitch, speech rhythm, tonal variations, breathing brakes.

According to yet other embodiments of the present invention, providing user data may comprise: operating wired or wireless communication utility and identifying one or more sensor units; obtaining physiological data via said wired or wireless communication utility, said physiological data comprising at least one of user biomedical and activity data from said one or more sensory units; using said one or more processors for processing said physiological data and said personalized data and determining an updated user anxiety/wellbeing profile as relating to at least one of selected groups of predetermined profiles.

The present method may further comprise: periodically obtaining updated physiological data from said one or more sensor units and processing said updated physiological data in accordance with a current user anxiety/wellbeing profile; and determining an updated user anxiety/wellbeing profile in accordance with said updated physiological data.

According to some embodiments, the method may further comprise determining a level of variation between current and updated user anxiety/wellbeing profile and generating variation data indicative of change in user anxiety/wellbeing profile.

The method may also comprise processing the variation data and consumption data indicative of consumption of one or more of said personalized nutrition additive items and transmitting data indicative of said variation data with respect to identity and time of consumption of the one or more of said personalized nutrition additive items to a follow up center for maintaining data on effect of said personalized nutrition additive items.

According to some embodiments, the physiological data may comprise one or more data pieces selected from: current heart rate, average heart rate, peak heart rate, rest heart rate, heart rate variability, respiratory rate, blood pressure, and blood oxygen saturation. Alternatively or additionally, the physiological data may comprise one or more data piece selected from: step count, stride length, walking pace, and activity measure. Further, the physiological data may comprise one or more data pieces selected from: sleep duration, sleep cycle length, number of sleep cycles in a night, duration of deep sleep, duration of REM sleep and sleep interruptions.

According to some embodiments, the method may further comprise generating user data request and receiving indication data on consumption of personalized nutrition additive items, said indication data comprising at least one of item identity and time of consumption.

According to some embodiments, the method may further comprise: storing data indicative of said current user anxiety/wellbeing profile; periodically generating an update request for a user to provide updated user personalized data indicative of at least one of user behavior and personal preferences; processing said updated user personalized data and determining an updated user anxiety/wellbeing profile; obtaining from a storage utility data indicative of current user anxiety/wellbeing profile, and processing said updated and current user anxiety/wellbeing profile for determining trend and level of variation in user anxiety/wellbeing profile; updating said current user anxiety/wellbeing profile and determining a recommendation on an updated collection of personalized nutrition additive items in accordance with the user anxiety/wellbeing profile and generating a corresponding provision request and transmitting said updated provision request via a communication network to a designated supply management utility.

Generally, according to some embodiments, the user anxiety/wellbeing profile is comprised of a nocturnal anxiety/wellbeing profile and a diurnal anxiety/wellbeing profile. The user anxiety/wellbeing profile may be indicative of user stress type and level. The user anxiety/wellbeing profile may comprise data indicative of user score in at least one measure selected from: sleep disorder, calmness, relaxation, focus, energetic-ness, and comfort.

According to some embodiments, the collection of personalized nutrition additive items comprises a selection of nutrition additive items (edibles) comprising selected profiles of active ingredients, selected for affecting one or more parameters of user anxiety/wellbeing profile. The active ingredients may comprise selected composition and dosage of Terpenes or terpenoids. Such terpenes may comprise one or more of fenugreek ((Trigonella foenum-graecum) or fenugreek extract (e.g., fenulife), and Phosphatidylserine (e.g., sharp ps), and may also include one or more of: a-Pinene, b-Pinene, b-Caryophyllene, Terpinolene, a-Humulene, a-Terpineol, 3A-Carene, Limonene, Linalool, a-Bisabolol, and trans-Ocimene. Additionally or alternatively, the active ingredients may comprise selected dosages of at least one of Cannabidiol (CBD) and Tetrahydrocannabinol (THC). Further additionally or alternatively, the active ingredients may comprise one or more herbs selected or extracted from: green tea, lemon balm, valerian, hops, guarana, chamomile, ginger, passionflower, ginseng, sage, rosemary, artichoke, and oats or green oats such as Avena sativa (e.g., neuravena).

According to yet some additional embodiments of the present invention, operating a user interface unit and generating a request for the user to provide user personalized data indicative of at least one of user behavior and personal preferences may comprise providing the user with a preselected questionnaire comprising a selected number of questions relating to personal behavior profile; the method further comprising operating a user facing camera and collecting video image data indicative of user activity at the time of answering said questionnaire; storing said video image data and said user personalized data for additional processing. The method may further comprise processing said video image data in accordance with user answers to said questionnaire and determining one or more physiological data pieces comprising, e.g., eye movement pattern.

According to one other broad aspect, the present invention provides a computer program storage device readable by machine, tangibly embodying a program of instructions executable by the machine to perform a method comprising: providing user data comprising at least one of user psychological data and user physiological data; using one or more processors for processing said user data and determining a current user anxiety/wellbeing profile comprising user score in one or more selected user anxiety/wellbeing parameters; determining a recommendation on a first collection of personalized nutrition additive items (edibles) in accordance with said user anxiety/wellbeing profile; and generating a provision request indicating said first collection of personalized nutrition additive items to be provided to the user and transmitting said provision request via a communication network to designated supply management utility.

According to an additional broad aspect, the present invention provides a computer program product comprising a computer useable medium having computer readable program code embodied therein the computer program product comprising: computer readable program code for causing the computer to provide user data comprising at least one of user psychological data and user physiological data; computer readable program code for causing the computer to use one or more processors for processing said user data and determining a current user anxiety/wellbeing profile comprising user score in one or more selected user anxiety/wellbeing parameters; computer readable program code for causing the computer to determine recommendation on a first collection of personalized nutrition additive items (edibles) in accordance with said user anxiety/wellbeing profile; computer readable program code for causing the computer to generate a provision request indicating said first collection of personalized nutrition additive items to be provided to the user and transmitting said provision request via a communication network to a designated supply management utility. According to one other broad aspect, the present invention provides a computer program comprising computer program code means for performing all the steps of the methods described herein when said program is run on a computer. The computer program may be embodied on a computer readable medium.

According to yet another broad aspect, the present invention provides a package comprising a collection of personalized nutrition additive items associated with a user anxiety/wellbeing profile, and instructions for selecting an item from the collection of items, the instructions are provided by a method described above.

The package may comprise a selected collection of personalized nutrition additive items associated with a user anxiety/wellbeing profile, and access code for operating a dedicated computer program product as described above.

### BRIEF DESCRIPTION OF THE DRAWINGS

In order to better understand the subject matter that is disclosed herein and to exemplify how it may be carried out in practice, embodiments will now be described, by way of non-limiting example only, with reference to the accompanying drawings, in which:

FIG. 1 illustrates general operation of a technique according to some embodiments of the invention;

FIG. 2 exemplifies selected wellbeing aspects according to some embodiments of the present invention;

FIG. 3A illustrates operation of a technique according to some embodiments of the invention;

FIG. 3B schematically illustrates a system using an interactive questionnaire for calculating a wellbeing profile of a user, according to embodiments of the invention;

FIG. 3C illustrates a method for using an interactive questionnaire for calculating a wellbeing profile of a user, according to embodiments of the invention;

FIG. 3D illustrates a method for presenting achievable goals to a user, according to embodiments of the invention;

FIG. 3E illustrates a method for determining sleep quality of a user, according to embodiments of the invention;

FIG. 3F schematically illustrates a method for determining a recommended stress zone for a user, according to embodiments of the invention;

FIGS. 4A and 4B exemplify an electronic device including one or more sensors and communication modules, the device suitable for operating a technique according to some embodiments of the invention; FIG. 4A shows a front side of the device and FIG. 4B shows a rear side of the device; and

FIG. 5 exemplifies various parameters used for determining a wellbeing profile of a user according to some embodiments of the invention.

### DETAILED DESCRIPTION

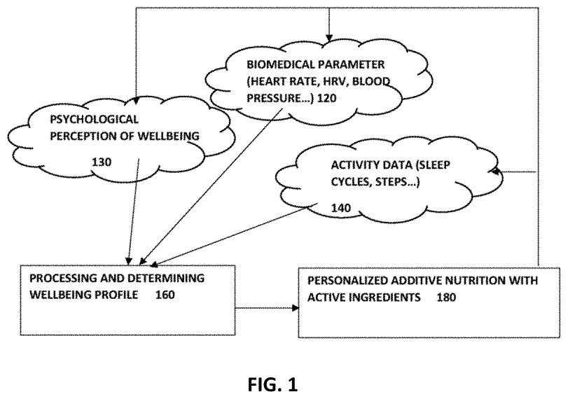

As indicated above, embodiments of the invention provide nutrition additive items, selected from a predefined collection of nutrition items, designed to address a user personal anxiety/wellbeing profile. Reference is made to FIG. 1, which generally exemplifies embodiments of the invention. As shown in FIG. 1, different types of user personal data may be used, including e.g., user physiological or biomedical parameters 120, user psychological perception of his own wellbeing 130, and user activity data 140. The user personal data is provided to a computer system (either local or remote computer system) for processing and determining a user wellbeing profile 160, which may generally relate to stress or anxiety of the user. Based on the user wellbeing profile, the processing includes determining a recommended collection of nutrition additive items 180 selected in accordance with the personal wellbeing profile. The recommended collection of nutrition additive items is selected based on one or more anxiety parameters to include nutrition additive items having one or more active ingredients associated with the one or more anxiety parameters.

In this connection, determining the user wellbeing profile 160 may include processing physiological data (e.g., physiological or biomedical parameters 120) for determining an anxiety level and/or determining a variation in anxiety level throughout a day. Similarly, an anxiety level and/or variation in anxiety level can be determined based on specific activities of the user. The anxiety data determined based on physiological data and/or activities of the user, may be correlated with psychological data (e.g., determined from user input) on type and level of anxiety. This correlation enables the processing according to embodiments of the invention, to differentiate between types of anxiety and improve the anxiety profile compared to the anxiety profile constructed from physiological data when take separately.

Generally, embodiments of the invention may further utilize follow-up on user consumption of the selected nutrition additive items and periodic update of the user wellbeing profile. This update of user wellbeing profile may typically take into account the effects of the selected nutrition additive items, as well as general variation of user anxiety or wellbeing profile caused by day-to-day life, health issues and any other cause that may affect the user.

To this end, embodiments of the invention may be operated through a software application running on a computer processing unit including one or more computer processors. For example, techniques according to embodiments of the invention may be operated as a smartphone application, internet page interface as well as combined cloud application using input through different computing systems and selected processing. Further, embodiments of the invention may also utilize a selected network communication channel, enabling communication with one or more provision centers for providing a personalized selection of nutrition additive items, namely and for providing instructions on selected nutrition additive items to be provided to the user based on the determined needs of each user.

The personalized selection of nutrition additive items may generally be selected from a predefined collection of nutrition items such as snack bars, shakes, herbs additives, or other selected nutrition items. The collection of nutrition items includes selected profiles of active ingredients selected to affect wellbeing aspects of users. The nutrition additive items may include selected active ingredients such as selected terpenes or terpenoids, e.g., obtained from Cannabis. Such terpenes may include one or more of fenugreek ((Trigonella foenum-graecum) or fenugreek extract (e.g., fenulife), and Phosphatidylserine (e.g., sharp ps). The active ingredients may also include selected amounts of Cannabidiol (CBD) and/or Tetrahydrocannabinol (THC); the active ingredients may also include selected herbs or other known active ingredients such as: green tea, lemon balm, valerian, hops, guarana, chamomile, ginger, passion flower, ginseng, sage, rosemary, artichoke, and oats or green oats such as Avena sativa (e.g., neuravena). The nutrition additive items are made with selected ingredient profiles for improving user wellbeing in selected aspects.

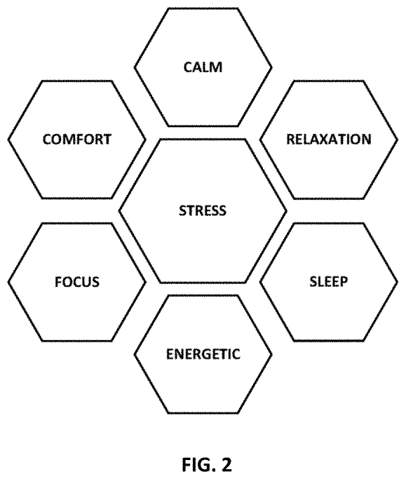

The nutrition additive items may generally include nutrition additive items having active ingredient combinations selected to improve different aspects of user anxiety. More specifically, the selection of nutrition additive items may utilize predefined formulations of active ingredients selected for improving selected aspects of user wellbeing as exemplified in FIG. 2.

As shown in FIG. 2, user wellbeing may be associated with a certain anxiety/stress level and different aspects of manifestation of stress in each individual user. To simplify user profiling, stress may be separated to six aspects relating to either diurnal or nocturnal behaviors including for example:

Calm, or calmness—relating to tranquility and peaceful mental state at nighttime;

Relaxation—relating to low tension state of being, typically during low-tension hours during the day;

Comfort—physiological and emotional comfort and feel at ease, generally during daytime;

Focus—ability to concentrate, relating to daytime and specifically to high activity/work time;

Energetic—level pf physiological and mental energy, relating to daytime and specifically to high activity/work time;

Sleep—sleep quality is associated with a number and duration of sleep cycles, waking up episodes throughout the night, sleep duration, and the time needed to fall asleep.

Generally, the set of parameters defining a user wellbeing profile may be used, according to embodiments of the invention, based on the different types of nutrition additive items as well as in determining parameters of user wellbeing profile. Thus, the collection of nutrition additive items may include item types (e.g., snack bar, shake, shake powder etc.) having selected formulations directed at the above indicated wellbeing aspects. The items' formulations are generally determined based on research on activity of the different active materials used in each type nutrition additive item and synergic effects of the active materials.

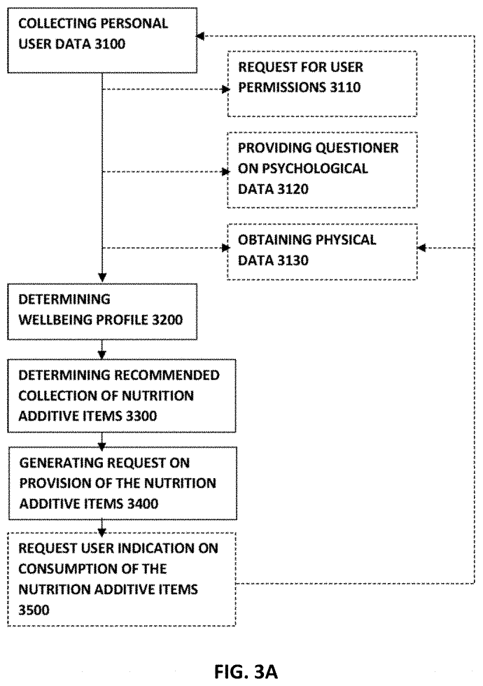

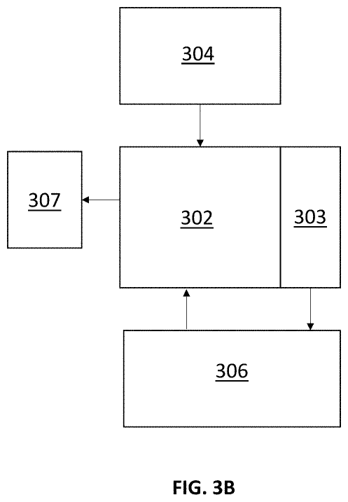

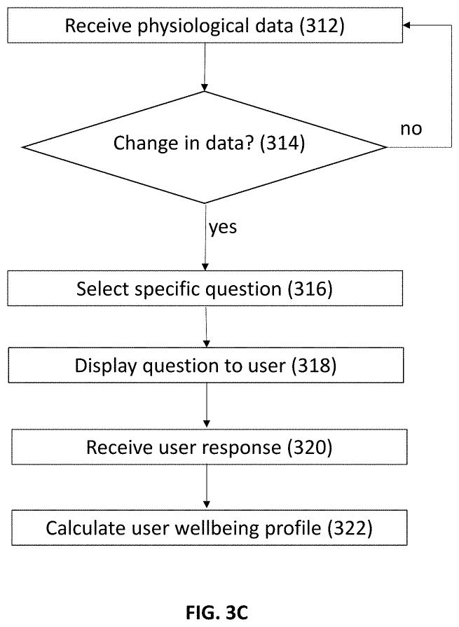

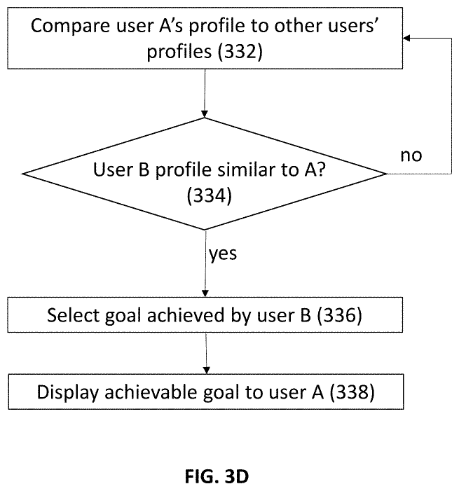

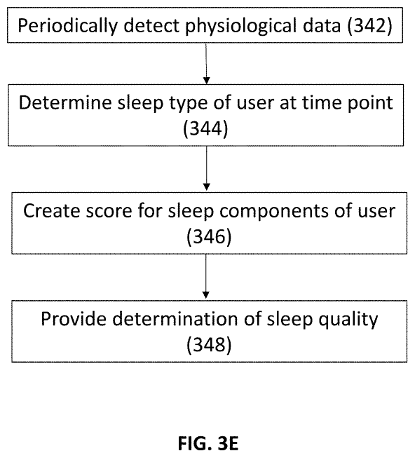

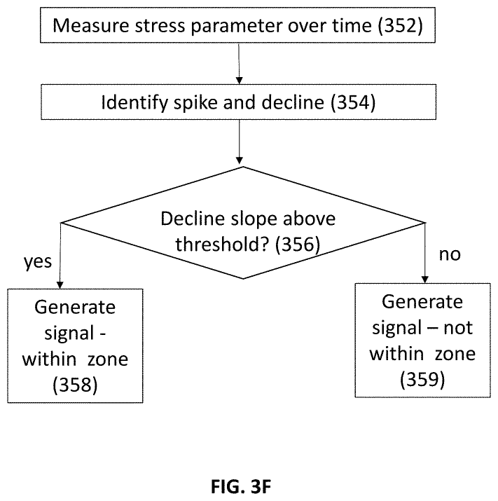

As indicated above, embodiments of the invention utilize collecting and processing personal data from the different users for determining personalized collection of nutrition additive items tailored for the user's wellbeing profile. Reference is made to FIG. 3A illustrating operation of a technique according to some embodiments. As shown, for personalization of the collection of nutrition additive items, the technique utilizes collection of personal data of the user 3100. The technique further utilizes the personal data for determining a wellbeing profile of the user 3200 based on a selected number of wellbeing aspects and determines a corresponding recommendation for a collection of nutrition additive items for the user 3300. Generally, the technique further generates a request, e.g., from a provision center, to provide the respective user with the recommended set of nutrition additive items 3400 for a selected period (e.g., package of items for a month's consumption). To further monitor and improve personalization, the technique may further include providing the user with a request for indications on use of the nutrition additive items 3500. Generally, such indications enable refined collection of user personal data 3100 for monitoring of the user wellbeing and operating within a cycle determined at least partially by selected periodicity of the request for provision of the nutrition additive items 3400 to the user.

Generally, collection of personal data 3100 may be associated with providing the user a request for permission for collection of personal data 3110. Such permission may typically be directed to data provided directly by the user, and may also include a request for permission to access data collected by one or more sensors that may be in communication with the electronic device running the technique (e.g., accelerometers, camera), third party sensors (e.g., heart rate sensors, watch, etc.) as well as external databases such as social network, activity monitoring network, etc.

Further, collection of personal 3100 data may generally include collection of psychological data and collection of physiological data of the user. For example, the technique may include providing the user with a questionnaire 3120 relating to user perception of his wellbeing profile, user activities, feelings, mood, etc. Further, the personal data may generally include obtaining physiological data 3130, collected via one or more sensors associated with the electronic device running the technique, third party sensors and/or obtained from selected databases or external applications. Typically, the collection of personal data 3100 includes a combination of collecting psychological data using a questionnaire 3120 or series of user inputs on self-perception of anxiety and obtaining physiological data 3130. This is based on the inventor's understanding that interpretation of physiological data to determine stress levels may generally be inadequate and that user indication of anxiety may provide valuable data for differentiating between activity levels and stress/anxiety.

In another embodiment, stress levels may be more accurately determined by creating an interactive questionnaire for user input regarding psychological data, based on physiological data. As schematically illustrated in FIG. 3B, creating the interactive questionnaire for determining a user wellbeing profile, may be carried out in a computer implemented method which includes using a processor 302 to detect a change in physiological data of the user, based on input from a sensor unit 304. For example, the physiological data may include biomedical parameters and user activity. Other physiological data may be collected by sensor unit 304, as described herein, and used by processor 302 to detect a change in physiological data of the user. In accordance with the detected change in physiological data the processor 302 selects, possibly, from a questions database stored in memory 303, a specific question relating to psychological data of the user, and provides the specific question to the user, typically, via a user interface 306.

Psychological data typically includes perception of the user, namely, a subjective assessment by the user of the user's physiological and/or psychological state, and is provided based on cognitive and mental processes of the user. Thus, the specifically selected question may relate to at least one of user behavior, personal preferences, and feelings. Other questions relating to psychological data, e.g., as further described herein, may be presented to the user. The user may respond to the specific question via the user interface 306.

Processor 302 may then calculate a user wellbeing profile 307 based on the response from the user to the specific question.

In one example of the method, which is schematically illustrated in FIG. 3C, a processor receives physiological data of a user (step 312). For example, physiological data may be collected and transmitted to the processor by an activity/smart watch or accelerometer sensor worn by a user. Measurements of physiological data are typically collected periodically. The processor determines if a change in physiological data has occurred (step 314), e.g., by comparing consecutive data measurements to each other. If a change has occurred the processor selects a specific question (step 316) relating to psychological data of the user, the question selected in accordance with the determined change in physiological data. For example, physiological data relating to diurnal wellbeing parameters (such as relaxation, focus and comfort) may be collected by a smart watch worn by a user, at a few time points during the day. A change in one or more physiological data (such as one or more of elevated/reduced heart rate, reduced heart rate variability, increased sweating, increase/decrease in body temperature, etc.) compared with previously measured data, triggers a selection of (one or more) question relating to psychological data. The specific one or more question may be selected in accordance with the type of physiological data that has changed (e.g., data relating to heart beat, data related to breathing, data related to movement, data related to a certain body part, etc.) and/or in accordance with a combination of types of data and/or in accordance with the magnitude of change (e.g., if the change is above a threshold) and/or in accordance with other relevant criteria related to the change.

Once a specific question (or questions) has been selected, it is provided to the user (step 318), e.g., presented on a UI. The selected question relates to psychological data of the user, for example, the question relates to at least one of user behavior, personal preferences, and feelings.

In some embodiments, the specific question is selected based on a change in physiological data and based on previously obtained psychological data of the user.

For example, for both users A and B different (e.g., better) sleep patterns are measured than sleep patterns measured on a previous night (e.g., shorter time than average to reach deep sleep, less awaking intervals during the night than usual, full and stable sleep cycles during the night, etc.). The psychological data previously collected for user A shows that user A suffers from difficulty in focusing. Thus the questions selected for user A, based on the detected difference in sleep patterns, will refer to improvements in fields affected by sleep patterns and related to difficulty in focusing. For example, the selected questions may related to user A's ability to focus, maintain concentration, gain deeper thoughts, etc. Examples of specific questions presented to user A may include:
In the past week/yesterday/today I went to do something, and once I got there, I forgot what I needed to do
In the past week/yesterday/today I was restless and easily startled by noises

Suggested answers for example:—5 times or more—3-4 times—1-2 times—Maybe once—Never happened).

The psychological data previously collected for user B shows that user B normally suffers from muscle tension and physical discomfort. Thus, the questions selected for user B, based on the detected difference in sleep patterns, will be focused on specific effects such as muscle tension, jaw clenching, stomach or digestive problems.

Examples of specific questions presented to user B may include:
In the past week/yesterday/today . . . I was short of breath
In the past week/yesterday/today . . . My heart was pounding or racing
In the past week/yesterday/today . . . I felt like I was sweating more than usual
In the past week/yesterday/today . . . I felt like there was a lump in my throat
In the past week/yesterday/today . . . I had a tingling sensation in my extremities

suggested answers for example:—5 times or more—3-4 times—1-2 times—Maybe once—Never happened).

The specifically selected questions try to assess the connection the user makes between relief of the reported symptoms, and the changed physiological data (in this example, sleep pattern data). In some embodiments, the connection may be reflected to the user to increase awareness of the user.

Once the user's response to the specifically selected question is received (step 320) the response is used by the processor to calculate the user's wellbeing profile (step 322), e.g., to update an existing profile or to create a new profile.

As described herein, the user's wellbeing profile is created based on scores assigned to wellbeing parameters of the user. In some embodiments, the processor assigns a score to a wellbeing parameter based on the response from the user and calculates the user wellbeing profile using the score. In one example, a higher weight is assigned to a score of a recent response from the user than the weight of a score of a previous response from the user. In other examples, a score is assigned to a wellbeing parameter based on the contents of the response (e.g., if the response is positive or negative) or based on a rating by the user. Other relevant criteria may be used for assigning a score to a wellbeing parameter of the user, in accordance with the user's response.

In some embodiments, the wellbeing parameter is associated with stress or anxiety. The wellbeing parameter may be associated with a stress type based on the Diagnostic & Statistical Manual of Mental Disorders.

Methods according to embodiments of the invention may be used to determine variations of stress level of the user throughout a day and may display, e.g., via the UI, an indication of the variations of stress level of the user, throughout the day. Thus, methods according to embodiments of the invention may be used to increase awareness of the user and assist in modifying habits and behavior to improve wellbeing of the user.

Methods according to embodiments of the invention may also be used to assist the user in achieving goals related wellbeing of the user, e.g., goals related to behavior, habits, feelings, etc. In one embodiment, which is schematically illustrated in FIG. 3D, user A's wellbeing profile is compared to wellbeing profiles of other users (step 332), to detect a similar profile. If (in step 334) a similar profile (or group of profiles) is found, e.g., if user B's profile is determined to be similar (e.g., above a threshold) to user A's profile, then a goal achieved by user B is selected (step 336) and the goal is displayed to user A as an achievable goal (step 338).

As discussed herein, a recommendation to the user for anxiety relieving nutrition additive items may be generated in accordance with the user wellbeing profile. Methods according to embodiments of the invention may include generating a provision request for the nutrition additive items to be provided to the user and transmitting the provision request via a communication network to a designated supply management utility.

In some embodiments, indication data on consumption of the nutrition additive items may be received. The indication data may include at least one of item identity and time of consumption. A variation in the user wellbeing profile is determined and the data on consumption of the nutrition additive items is compared to the variation in the user wellbeing profile to obtain an estimation of the effect of the nutrition additive items on the wellbeing of the user.

As discussed above, in some embodiments a specific psychological question is presented to a user in accordance with a change in physiological data of the user when the change relates to diurnal wellbeing parameters. In this case the processor may be also used to detect sleep related physiological data of the user, (e.g., based on input from one or more sensor unit) and a score for a nocturnal wellbeing parameter may be determined based on the sleep related physiological data. This score may be used (possibly in addition to other scores) to calculate the user wellbeing profile. In this embodiment, the processor may detect sleep related physiological data compatible with sleep components of the Pittsburgh Sleep Quality Index (PSQI), as further detailed below.

The Pittsburgh Sleep Quality Index (PSQI) is an index (in the form of a self-report questionnaire) developed to measure quality of sleep. The index consists of 19 individual measures, creating seven sleep components that produce one global score. The components include subjective sleep quality, sleep latency, sleep duration, habitual sleep efficiency, sleep disturbances, use of sleep medication, and daytime dysfunction. Although the questions are brief and easy to understand and answer, they still require participation of the user and provide a measure based on typically subjective information and based on what the user may or may not remember.

Embodiments of the invention enable obtaining scores compatible with the PSQI in an automated method using substantially real-time data and without relying on user subjective input. Thus, the method according to embodiments of the invention provides a more accurate and easy way to determine sleep quality than the methods used to date.

In one embodiment, which is schematically illustrated in FIG. 3E, a computer implemented method is used to determine sleep quality of a user. The method includes using a processor to detect sleep related physiological data of the user in different time points (step 342), e.g., based on input from a sensor unit collecting and/or transmitting the data once a minute. In step 344 the processor determines, from the sleep related physiological data, a sleep type of the user (such as Deep sleep, REM, Light Sleep or awake) at each of the time points (e.g., in every minute) and based on the sleep type at each time point, the processor creates a score for sleep components of the user (step 346). Typically, the sleep components are as defined by PSQI. The score may then be used to provide a determination of sleep quality of the user (step 348) and the determination may be compatible with the PSQI.

One or more sensors can be used to collect physiological data such as level of blood oxidation, level of alcohol in the blood, body temperature and movement of body parts. The sensor (or another sensor) may additionally collect environmental data such as room temperature, level of light in the room, noise level in the room, use of mobile phone, etc. One or a combination of such data can be used by the processor to create the score for the sleep components of the user.

Improvement in wellbeing parameters of the user may be assessed in light of the determination of sleep quality, according to embodiments of the invention. For example, for a user reporting a feeling of lack of stamina, a change (e.g., improvement) in sleep quality as determined based on physiological and other data provided from sensors, may cause a question regarding stamina to be presented to the user. If the user responds to this specific question by indicating a better feeling of stamina, then it may be concluded that the improved feeling is due to improved sleep quality. If the user responds by indicating a continued lack of stamina, then it may be concluded that other physiological parameters are responsible for the lack of stamina. These conclusions may be used to improve, for example, selection and personalization of recommended nutrition additive items. Alternatively or in addition, these conclusions may be reflected to the user to increase awareness of the user to the effect of physiological and/or environmental occurrences on the psychological wellbeing of the user, and may assist in modifying habits and behavior of the user to improve his/her wellbeing.

In another embodiment of the invention, stress parameters are monitored and reflected to the user to assist the user in understanding and aiming for the behavior and activities that optimally enable the user to contain and deal with stress.

Generally, stress shouldn't be “as low as possible” since it is essential for people's healthy functioning. For instance, stress helps people wake up in the morning and enables people to become focused. It would be advantageous for each user to find the right level of stress that helps him/her to function well, without negative side effects. Thus, some embodiments of the invention are directed at creating a recommended zone for each user, in which the user's stress burden is optimal.

In one embodiment, which is exemplified in FIG. 3F, an optimal or recommended “stress zone” is detected for a user. Detecting a recommended stress zone includes periodically measuring a stress parameter and optionally generating a map (e.g., a graph) of the stress parameter over time.

The stress parameter may include one or more physiological measurements, such as, heart rate variance and possibly additional measurements such as user activity, user motion and/or if a user is sleeping, the conductivity of the user's skin.

Measurements of the stress parameter are collected periodically, e.g., every few minutes (such as, once every 3 minutes or less, e.g., once every two minutes or once a minute).

In one embodiment, a method for determining a recommended stress zone for a user includes obtaining a value of a stress parameter of the user over time (step 352). In step 354 a spike and subsequent decline in the value of the stress parameter are identified. A desired behavior of a stress parameter over time includes a quick decline following a spike in the stress parameter. A quick decline of the stress parameter indicates that the user was able to contain the stress and calm down quickly. The higher the percentage slope of the decline (the steepness of the slope), the better the user is able to contain the stress and go on with his/her regular activities. Thus, a recommended zone for the user may be determined based on the slope of the decline and a signal indicating the user is within the recommended zone, may be generated based on the slope of the decline. In one embodiment, if the percentage slope of the decline is above a threshold (step 356) a signal is generated (step 358) to provide an indication to the user that the user is within the recommended zone. However, if the percentage slope is below the threshold (which may indicate a prolonged period of calming down by the user), a signal is generated (step 359) to provide an indication to the user that the user is not within the recommended zone.

Different users react differently to stress such that each user may have different sized spikes. Therefore, in some embodiments, the threshold, by which to assess steepness of the decline of a spike, is based on the user's own spikes, typically, based on an average (or other statistic) of several of the user's spikes. Thus, if the percentage slope of a decline is larger than the percentage slope of its spike (or larger than an average or other statistic of the percentage slope of several of the user's spikes) then a signal is generated to provide an indication to the user that the user is within the recommended zone, whereas, if the percentage slope of the decline is lower than the percentage slope of its spike, then a signal is generated to provide an indication to the user that the user is not within the recommended zone.

Using this embodiment, an understanding may be provided of which stress situations the user is able to contain and which less so. Based on this understanding, recommendations (e.g., as described herein) may be provided to the user and/or the user may alter his activities and behavior accordingly. Goals and reports generated based on determination of a stress zone may help the user aim and measure her/himself and embrace a routine that helps the user reach the recommended zone.

Thus, in some embodiments, a method may include possibly selecting, in accordance with a detected change in physiological data, a specific question relating to psychological data of the user and providing the specific question to the user (e.g., via a user interface), as well as obtaining a value of a stress parameter of the user, over time, identifying a spike and a subsequent decline of the value of the stress parameter and displaying a recommendation to the user based on the percentage slope of the decline.

The zone determined according to embodiments of the invention, is personalized to each user and may be recalculated based on the user's responses to questions on one hand, and the physiological data periodically collect from the user.

As indicated above, techniques according to embodiments of the invention may be implemented by a computer system including one or more processors, storage (memory) utility and network communication utility. For example, techniques may be operated as smartphone applications or a web page application.

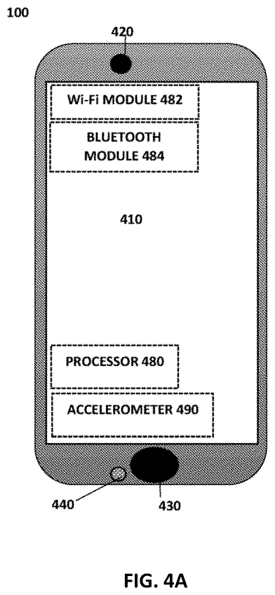

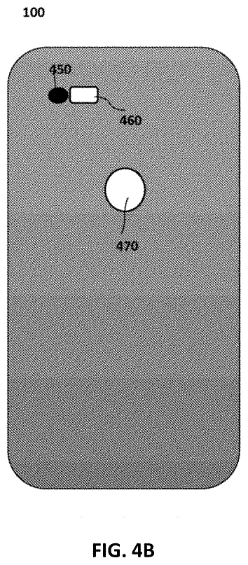

Reference is made to FIGS. 4A and 4B exemplifying an electronic device 100 (e.g., a smartphone device) capable of operating technique according to embodiments of the invention. Device 100 may be, e.g., loaded with software code that when operated by the processor 480 performs techniques according to embodiments of the invention. Device 100 may include a user interface including screen 410 (e.g., touchscreen) and keyboard of buttons 430 when used separately or in exchange of touchscreen, and speaker/microphone 440. Further, one or more sensors may be built into the electronic device 100, such as front facing camera 420, rear facing camera 450 with or without LED light source 460, fingerprint sensor 470 and accelerometer 490 or other location sensors (such as GPS). Additional sensors may further be used but are not specifically shown herein. As shown, the device 100 typically also includes communication modules such as Wi-Fi module 482 and Bluetooth module 484.

As indicated above, the user interface, including screen 410 and buttons/keys 430, typically provides user input and output communication. Further, the user interface, including screen and speaker, may be utilized for generating a request to a user to provide user personal data, and to request user permission when such permission in needed. To provide psychological data, embodiments of the invention generally provide the user with a questionnaire relating to user behavior, feelings, and expectations. For example, the questionnaire may be formed by a number of questions relating to anxiety related habits/feelings such as: desire to change/reduce stress level; user symptoms when under stress; preferences on use of technology; frequency of physiological activity; health tendencies; decision making; personal description; frequency of feeling nervous or anxious; general feeling; commuting habits; sleep quality; disorientation or discomfort during day and during sleep; ability to focus on tasks; and other aspects for providing a psychological profile of the user. The electronic device 100 may be operated for receiving input of user responses to the questionnaire and for providing the input data for processing to determine a user wellbeing profile. Generally, the user input in response to the questionnaire may be stored in local or remote storage/memory (not specifically shown) for future comparison and/or sent for processing for determining user wellbeing profile.

The processing may utilize predetermined, generally non-linear, summation of user responses. Generally, the wellbeing profile may be determined as scores of one or more wellbeing aspects, e.g., as described above, scores of aspects such as calm, comfort, relaxation, sleep, energy, and focus. For example, given that the user response to each question Qi can be represented by a number qi within a predetermined range for each question (e.g., 1-2 for some questions, 1-5 for other questions and 1-10 for some further questions), the total wellbeing score Aₗ for each aspect/may be determined by:

Aₗ₌Σwₗⁱqⁱ,

where wₗⁱ may be a selected coefficient and may also be a function of qⱼ for j≠i. Thus, the wellbeing score may generally be a nonlinear sum of the user input data pieces (responses to the questionnaire). In some embodiments, the wellbeing profile of the user is determined by processing at a local processor, whereas in some further embodiments, the processing may be remote processing, and may include further analysis using one or more pre-trained computer learning machine techniques.

In addition to collection of psychological data, embodiments of the invention may collect physiological data of the user. To this end, one or more of the sensors included in the electronic device 100 may be utilized for collecting data on eye movement, physiological activity, step count, hands movement while holding the device (e.g., device 100), etc. Additionally or alternatively, user activity data may be obtained from one or more third party sensors or databases. Additional activity monitoring applications installed on the device 100 may be utilized and some activity data may be stored on device 100, or in a remote storage location. To this end, embodiments of the invention may acquire biomedical data such as heart rate, heart rate variability (HRV), maximal heart rate, rest heart rate, breathing sounds, etc., and activity data such as number of steps, step length, eye movement, meditation, sleep duration and cycles, general activity type, etc. Generally, a technique may request user input on type of activity tracking sensors or applications used, or obtain such data based on already installed applications on the device 100. The technique may further request access to collected activity information from sensors or applications for collecting user physiological data.

For example, a technique may operate front facing camera 420 for collecting image video data of the user while responding to questionnaire. The collected video data may be processed, locally or at a remote processing utility, for determining user stress related activity at the time of answering the questionnaire. This may be based, e.g., on rapid eye movement, focusing, rate of answering the questionnaire, etc.

In some other examples, a user's voice via microphone 440, while performing regular telephone conversations or at selected specific times, may be recorded. User voice recordings may be processed for determining stress related voice variations such as hoarseness, tone variations, pitch voice, speech rhythm, tonal variations, breathing brakes, etc. Further, the processing may provide data indicative of heat rate variability based on changes in the user's voice.

In some additional examples, fingerprint sensor 470 and/or rear facing camera 450 combined with light source 460 may be utilized for collecting heart rate and sweat data from the user. For example, the user may be instructed to place a finger on one of the fingerprint sensors 470 or the rear facing camera 450, while techniques according to embodiments of the invention operate the respective sensor for collecting variations in light or electrical activity (for the fingerprint sensor) indicative of user heart rate changes and/or sweat level. Such parameters are typically known to be related at times to stress levels of the user.

The physiological data of the user may be used for determining user wellbeing profile in combination with the psychological data. More specifically, selected physiological data pieces, and/or activity data pieces may be used within the scoring for wellbeing aspects as indicated above. For example, the physiological and activity data of the user may be arranged in selected number of data pieces Pᵢ where each data piece may have the score pᵢ relating to association of the relevant activity to wellbeing or stress of users. The total wellbeing score Aₗ for each aspect l may be determined by:

Aₗ₌Σwₗⁱqⁱ+Σwₗⁿpₙ

where wₗⁱ and wₗⁿ are generally functions of qⱼ and pₘ for j≠i and m≠n.

Thus, an initial wellbeing profile of the user may be determined using partial data being psychological data and/or physiological data. The wellbeing profile may be further improved while collecting additional data on the user, by monitoring user activity over time, providing repeating questionnaires, obtaining activity data from additional sources, etc. Generally, a predetermined number and type of data pieces (e.g., either psychological or physiological/activity relates) may be required for marking the wellbeing profile of the user as sufficient for generating recommended collection of nutrition additive items. After acquiring personal data over the required minimum, embodiments of the invention may further improve the wellbeing profile of the user, and the corresponding recommendation accordingly.

As indicated above, embodiments of the invention utilize the determined wellbeing profile of the user for generating a recommendation of collection of nutrition additive items from a predetermined selection of nutrition additive items formulated for improving one or more wellbeing aspects.

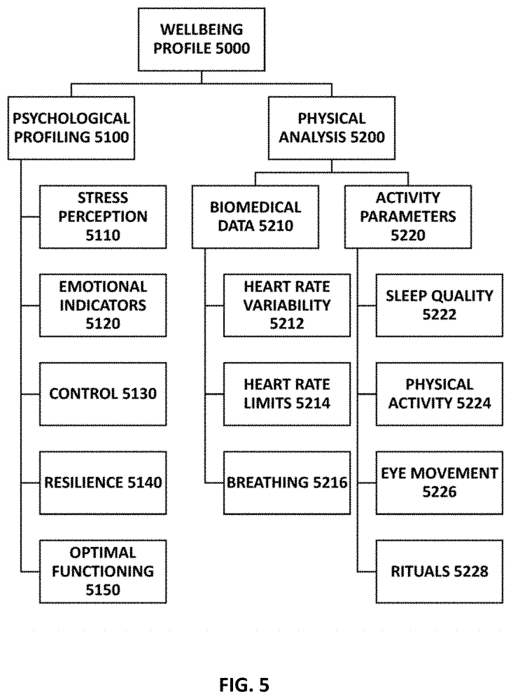

Reference is made to FIG. 5 exemplifying various parameters used for determining a wellbeing profile 5000 of the user. As indicated above, the profile is generally constructed of scores in one or more selected wellbeing aspects. The wellbeing profile is generally determined using one or more types of user data including psychological profiling 5100 (psychological data), physical analysis 5200 (physiological data) generally based on biomedical parameters 5210, and activity parameters 5220. Generally, the technique described in FIG. 5 may determine a wellbeing profile based on one or more of these types of data pieces, where the more data used, the better the profiling of the user.

The psychological profiling of the user, typically obtained through a questionnaire, may include user perception of stress 5110, emotional indicators 5120, user control 5130 (or perception of control), resilience 5140, and optimal functioning 5150 relating to situations of optimal functioning and user perception of such situations.

The physical analysis of the user may include analysis of biomedical data 5210 and activity parameters 5220. The biomedical data 5210 may include heart rate variability 5212 associated with variation in heart rate between inhale and exhale, limits of heart rate 5214 through the day, data on breathing 5216, and may include additional biomedical parameters such as blood pressure, blood oxygenation etc. The biomedical parameters may be determined based on average parameters, response to situations (e.g., parameters while answering the questionnaire), and/or maximal/minimal values, etc.

The user activity parameters 5220 may be determined based on activity data collected by one or more sensors (such as activity/smart watch, accelerometer sensor 490 of electronic device 100 in FIGS. 4A and 4B, etc.). The user activity may be based on one or more parameters including for example: sleep quality 5222 associated with sleep cycles, waking up, and time to fall asleep; physical activity 5224 based on amount of activity, number and rate of steps, etc.; eye movement 5226, e.g., while operating a phone and when focusing; and general rituals 5228 of the user.

In some configurations, the wellbeing profile may be determined using a pre trained learning machine (classifier) or based on selected predetermined formulae as indicated above. Such formulation by itself is determined by the inventors based on research on stress levels of various individuals.

As indicated above, the collection of nutrition additive items is formed by a selected set of nutrition items such as bars, cookies, jells, shakes, or shake powder, candies or any other type of nutrition items including one or more active ingredients whose profile is selected to address different aspects of user wellbeing. For example, the nutrition additive items may include different item types directed at improving user calmness, relaxation, comfort, focusing ability, energetic feel, and sleep quality, additional types may also be used. To simplify processing, the user wellbeing profile may be determined by determining scores in a selected number of wellbeing aspects corresponding to the types of nutrition additive items available. In some embodiments, the technique may operate to provide recommendations and suggest a request for provision of the nutrition additive items, at selected periodicity, e.g., once in a week, once in a month etc. In this case, the recommendation of collection of nutrition additive items may generally be based on wellbeing scores in the different aspects such that a user having low sleep score (relevant to low sleep quality) will receive recommendation on sleep improving items, and user having low focus score (relevant to difficulty to focus) will receive recommendation on focusing improving items.

For example, given a user wellbeing profile such as: calm 8; comfort 6; relaxation 7; focus 3; energy 2; sleep 9 (scores are 1-10 where 10 is difficulty in the relevant aspect and 1 means no difficulty). Such a user may be characterized as having difficulty in sleeping or in being calm, and relatively high energy and focusing ability. Such a user may receive recommendation on 30 nutrition additive items to be used within a month (e.g., one a day) based on the scoring parameter normalized to the total number of items. Thus, the recommendation may be 7 items relating to calm; 5 items relating to comfort; 6 items relating to relaxation; 3 items relating to focus; 1 item relating to energy and 8 items relating to sleep. Upon determining a recommended set of nutrition additive items, the processor 480 may operate to generate a request and send the request to a provision center (e.g., via network communication or telephone line). Generally, in some configurations, the processor 480 may generate an indication to the user requesting confirmation on the order and approval of relevant payment.

The nutrition additive items are generally pre-prepared using standard recipes and including one or more active ingredients selected for addressing wellbeing issues as indicated above. Such active ingredients may typically include selected terpenes, as well as additional active ingredients such as CBD, THC, green tea, lemon balm, valerian, hops, guarana, chamomile, ginger, passion flower, ginseng, sage, rosemary, artichoke, oats or green oats such as Avena sativa (e.g., neuravena), fenugreek ((Trigonella foenum-graecum) or fenugreek extract (e.g., fenulife), and Phosphatidylserine (e.g., sharp ps).

Generally, terpenes are materials produced by plants for interactions with other organisms. Terpenes often protect plants against pathogens like mold, fungus, and bacteria, and can attract pollinating insects or repel herbivores. There are thousands of types of terpenes known across the plantae, while a small percentage of all terpenes have been identified. Various known terpenes are found in cannabis plants.

The therapeutic effect of Cannabis largely depends on the content of its pharmacologically active secondary metabolites, mainly phytocannabinoids, flavonoids, and terpenoids. Of the over 500 different compounds present in the Cannabis plant, currently more than 120 have been identified as phytocannabinoids, the unique bioactive compounds of the Cannabis plant, and approximately 200 identified terpenoids at various concentrations. The therapeutic effects of cannabis depend on specific terpenoids as well as synergistic effects with other active compounds in the plant. Terpenes of cannabis plants are typically simple mono and sesquiterpenes derived from two and three isoprene units. Cannabis produce and accumulate a terpene rich resin in glandular trichomes, which are most abundant on the surface of female inflorescences.

Terpenoids act on a verity of receptors and neurotransmitters. Some terpenoids act as serotonin uptake inhibitors, enhance norepinephrine activity, increase dopamine activity, and augment GABA. These activities have similarities to known medicines such as Prozac, various antidepressants, monoamine oxidase inhibitors, benzodiazepines, and others. Accordingly, selected combination of phytocannabinoids and terpenes provides complementary or synergistic results often referred to as the “entourage effect”. This is while there is evidence that terpenes alone can attribute to some of the physiological response specifically on the effects of cannabis on mood. The nutrition additive items as described herein may generally include selected amounts of terpenes, selected in accordance with inventors' research on effects of terpenes formulations on selected wellbeing aspect. The active ingredients may include terpenes of different types such as a-Pinene, b-Pinene, b-Caryophyllene, Terpinolene, a-Humulene, a-Terpineol, 3A-Carene, Limonene, Linalool, a-Bisabolol, and trans-Ocimene. Each type of nutrition additive item includes selected profile of the terpenes in accordance with the selected wellbeing aspect.

As indicated above, the present technique may further request the user to provide an indication on consumption of the nutrition additive items, including type of item and time of consumption. This data combined with general monitoring of physiological data and activity of the user may be further used for updating user wellbeing profile and the effects the recommended nutrition additive items have on each specific user. Periodic monitoring of the user physiological data, and at times of the psychological data, combined with indications of nutrition additive items' consumption may also be used for raising alerts in case certain medical or physiological parameters deteriorate over a predetermined threshold, thereby enabling to identify side effects and other medical conditions of the user that may require treatment or changes in treatment. Additionally, this may enable identifying reduced wellbeing in different aspects and finetuning the recommended nutrition additive items, or in certain cases, raising an alert to the user and/or to medical professionals as the case may be.

Thus, embodiments of the invention enable determining recommended one or more sets of nutrition additive items, selected from a predefined collection, and tailored to a personal wellbeing profile of the user. Techniques according to embodiments of the invention may be operated using a local processing unit (local processor) and/or utilize remote processing for certain processing activities.

What is claimed is:

1. A computer implemented method for determining a user wellbeing profile, the method comprising:
using a processor to detect a change in physiological data of the user, based on input from a sensor unit;
selecting, in accordance with the detected change in physiological data, a specific question relating to psychological data of the user;
providing the specific question to the user, via a user interface (UI); and
calculating the user wellbeing profile based on a response from the user to the specific question.

2. The method of claim 1 wherein the specific question relates to a subjective assessment by the user of a physiological state of the user.

3. The method of claim 1 comprising selecting the specific question based on previously obtained psychological data of the user.

4. The method of claim 1 wherein the physiological data comprises user biomedical parameters and user activity.

5. The method of claim 1 comprising:
assigning a score to a wellbeing parameter based on the response from the user; and
calculating the user wellbeing profile using the score.

6. The method of claim 5 comprising assigning a higher weight to a score of a recent response from the user than a weight of a score of a previous response from the user.

7. The method of claim 5 wherein the wellbeing parameter is associated with stress and wherein the method comprises determining variations of stress level of the user throughout a day.

8. The method of claim 7 comprising displaying via the UI an indication of the variations of stress level of the user throughout the day.

9. The method of claim 7 wherein the wellbeing parameter is associated with a stress type based on a Diagnostic & Statistical Manual of Mental Disorders.

10. The method of claim 1 comprising:
comparing the user wellbeing profile to wellbeing profiles of other users to detect a similar profile; and
displaying to the user an achievable goal based on goals achieved by a user of the similar profile.

11. The method of claim 1 comprising generating a recommendation to the user for anxiety relieving nutrition additive items in accordance with the user wellbeing profile.

12. The method of claim 11 comprising:
generating a provision request for the nutrition additive items to be provided to the user; and
transmitting said provision request via a communication network to a designated supply management utility.

13. The method of claim 11 comprising:
receiving indication data on consumption of the nutrition additive items;
determining a variation in the user wellbeing profile; and
comparing the data on consumption of the nutrition additive items to the variation in the user wellbeing profile to obtain an estimation of the effect of the nutrition additive items on wellbeing of the user.

14. The method of claim 1 comprising:
using the processor to detect sleep related physiological data of the user, based on input from the sensor unit;
determining a score for a nocturnal wellbeing parameter based on the sleep related physiological data; and
using the score to calculate the user wellbeing profile.

15. The method of claim 14 comprising using the processor to detect sleep related physiological data compatible with sleep components of Pittsburgh Sleep Quality Index (PSQI).

16. A computer implemented method for determining sleep quality of a user, the method comprising:
using a processor to detect sleep related physiological data of the user in different time periods, based on input from a sensor unit;
determining, from the sleep related physiological data, sleep type of the user at each of the time periods;
based on the sleep type at each time period, creating a score for sleep components of the user, the sleep components as defined by PSQI; and
using the scores to provide a determination of sleep quality of the user.

17. The method of claim 1 comprising:
obtaining a value of a stress parameter of the user, over time;
identifying a spike and a subsequent decline of the value of the stress parameter; and
displaying a recommendation to the user based on a percentage slope of the decline.

18. A method for determining a recommended stress zone for a user, the method comprising:
obtaining a value of a stress parameter of the user, over time;
identifying a spike and a subsequent decline of the value of the stress parameter; and
if a percentage slope of the decline is above a threshold, then generating a signal indicating the user is within the recommended stress zone.

19. The method of claim 18 wherein the threshold is based on one or more spikes identified for the user.
